(19) United States
(12) Patent Application Publication
(10) Pub. No.: US 2015/0297508 A1
(43) Pub. Date: Oct. 22, 2015

(54) Topical Formulation to Enhance Genital Sensation and Function

(71) Applicant: William Andriette, Roxbury, MA (US)

(72) Inventor: William Andriette, Roxbury, MA (US)

(21) Appl. No.: 14/692,555

(22) Filed: Apr. 21, 2015

Related U.S. Application Data
(60) Provisional application No. 61/981,932, filed on Apr. 21, 2014.

(51) Int. Cl. A61K 9/00; A61K 31/4525; A61K 31/7076; A61K 31/045; A61K 47/46; A61K 31/706; A61K 31/34; A61K 31/7084; A61K 31/375; A61K 31/198; A61K 31/7004; A61K 31/22

(52) U.S. Cl. CPC A61K 9/0014; A61K 31/7004; A61K 31/4525; A61K 31/7076; A61K 31/045; A61K 31/22; A61K 31/706; A61K 31/34; A61K 31/7084; A61K 31/375; A61K 31/198; A61K 47/46

(57) ABSTRACT

A topical formulation that functions to enhance genital sensation and function can be utilized by either male or female users. The topical formulation incorporates active compounds which function cooperatively to stimulate nerve activity, enhancing sensitivity, and facilitating tumescence in order to increase pleasure from physical stimulation and orgasms. It has incorporated various compounds that provide energy and support energy production in a cell, that stimulate nerve activity and sensitivity, that support blood flow and/or vasodilation to a bodily region, that support penetration and transport of active compounds, and that preserve the efficaciousness, effectively dilute, serve as a carrier.

The current application claims a priority to the U.S. Provisional Patent application Ser. No. 61/981,932 filed on Apr. 21, 2014.

### FIELD OF THE INVENTION

The present invention relates generally to a topical formulation. More specifically, the present invention is a topical formulation that is able to enhance sensation and function to the genital region.

### BACKGROUND OF THE INVENTION

Currently, there are many nutritional products and medications that are aimed for sexual enhancement. Sexual enhancement formulas are provided as a means of improving sensation, genital function (e.g., tumescence), and duration of sexual activity. Existing sexual enhancement formulas are most commonly provided as an orally administered compound that is ingested by a user. One of the disadvantages of orally-administered sexual enhancers is due to their delay between ingesting the sexual enhancement formula and the onset of its effects. The delayed onset of the effects can be caused by a variety of factors, including the user's own digestive system. As well, orally ingested enhancers—even when their efficacy is desired only in the genitals—are typically distributed more systemically in the body, where they are inefficacious or can produce side-effects.

It is therefore an aim of the present invention to provide a sexual enhancement formula as a topical formulation that is applied to the penis or clitoris before sexual activity. The topical formulation incorporates active compounds which function co-operatively to stimulate nerve activity, enhancing sensitivity, and to facilitate tumescence, and to heighten orgasmic peristaltic intensity in order to increase pleasure from physical stimulation and orgasms. The present invention is intended, primarily, for individual without sexual difficulties, but the topical formulation has been shown to be beneficial for those suffering from deficiency of sexual function.

### DETAIL DESCRIPTION OF THE INVENTION

The present invention is a topical formulation that is able to enhance genital sensation and function. The topical formulation is utilized by either male or female users. The topical formulation incorporates active compounds which function cooperatively to stimulate nerve activity, enhancing sensitivity, and to facilitate tumescence and heightened orgasmic peristaltic intensity in order to increase pleasure from physical stimulation and orgasms. The present invention incorporates compounds that provide energy and/or support energy production in a cell, that stimulate nerve activity and sensitivity, that support blood flow and/or vasodilation to a bodily region, that support transdermal or transmuscosal penetration and transport of active compounds, and that preserve the efficaciousness, effectively dilute, serve as carriers, and improve the tactile properties of the aforementioned active compounds. It should be noted that active compounds may provide overlapping functionality in the formulation resulting in compounds that support more than one function of the formulation.

The present invention incorporates compounds that provide energy and/or support energy production in a cell, such as a nerve cell or muscle cell. In one embodiment of the present invention, the topical formulation selects at least one compound from a group containing D-ribose, adenosine triphosphate (ATP), a reduced form of nicotinamide adenine dinucleotide (NADH), nicotinamide riboside, and creatine.

D-ribose is the right-handed enatiomer of the five-carbon sugar ribose, which is converted for use in the pentose phosphate pathway. ATP is a phosphorylated derivative of ribose that functions as an energy-rich compound that drives a majority of intracellular activity. NADH is another phosphorylated derivate of ribose that functions as an electron acceptor. Nicotinamide riboside, a niacin-related compound found in some foods, is a precursor to the oxidized form of NAD (NAD+), helps maintain intracellular concentration of NAD, can support neuroprotection, and mitochondrial and endothelial function. Creatine has been used to regenerate ATP under sudden conditions of high metabolic demand. The aforementioned compounds support energy production and efficiency in nerve and other cells of a user's genitalia. Specifically, nerve cells have high energy demands, particularly at their extremities, where they have high concentrations of mitochondria and high demand for mitochondrial ATP production. By providing and supporting energy production within nerve cells function and activity is improved.

The present invention incorporates nerve-stimulating compounds that, when utilized in conjunction with energy-providing and/or energy-production supporting compounds that up-regulate cells to a more metabolically active state, allow said nerve cells to upward cycle stimulation-enhancing sensations and activity. In the current embodiment of the present invention, the topical formulation uses 95% piperine in the form of a standardized extract from black pepper.

Piperine is an alkaloid derived from black pepper that provides a stimulating effect when topically applied. When applied to metabolically supported nerve cells, the topical formulation is able to increase sensitivity, as for example from sensations of sexual stimulation.

The present invention incorporates blood-flow enhancing and/or vasodilating compounds that improve tumescence and blood circulation to a user's genitals. It is known that tumescence is the engorgement of erectile tissue with blood. By increasing circulation and blood flow to a user's genital region, nutrients are delivered that assist in supporting higher metabolic activity to the applied region, supporting nerve and muscle function. In one embodiment of the present invention, the topical formulation utilizes at least one of blood flow enhancing and/or vasodilating compounds from the group containing ATP, NADH, nicotinamide robisode, diemethyl isosorbide, and a black pepper extract.

ATP has been shown to signal increased blood flow to a region of a user's body when found extracellularly. NADH has been observed to work as a vasodilator when found extracellularly. Nicotinamide riboside helps activate the protein Sirtuin1, which in turn promotes endothelial nitric oxide synthase, supportive of vasodilation. Dimethyl isosorbide, derived from corn sugar, is metabolized to produce isosorbide, which is a known vasodilating compound that is traditionally used to treat angina and congestive heart failure. Black pepper-has been shown to increase blood-flow and vasodilation when topically applied-and as well functions as thermogenic agent, providing a heating sensation to a user's skin, which stimulates vasodilation. Through the inclusion of the aforementioned compounds, the topical formulation is able increase circulation to support cells and tissue with high metabolic activity while additionally providing a tumescence effect to erectile tissue in the corpora cavernosa of the penis and in the clitoris.

The present invention incorporates compounds that assist in the dermal and mucosal absorption and penetration of active compounds. In the current embodiment of the present invention, the topical formulation utilizes at least one compound from a group of dermal and mucosal absorption- and penetration-enhancing compounds containing piperine, bisabolol, dimethyl isosorbide, and medium-chain triglycerides.

Piperine has been experimentally observed to facilitate penetration and absorption of active compounds across dermal and mucosal membranes. Bisabolol is an essential oil primarily found in German chamomile that has been seen to enhance precutaneous absorption of certain large molecules. Dimethyl isosorbide functions as an epidermal penetration enhancer. Medium-chain triglycerides (MCT) have a high absorption rate with the body, which facilitates transportation of various compounds across dermal and muscosal surfaces. Through the incorporation of dermal and muscosal absorption- and penetration-enhancing compounds, the topical formulation is able to reduce the quantity of the active compounds needed to achieve an intended effect. It should be noted while the absorption- and penetration-enhancing compounds are described as being able to enhance both transdermal and transmucosal absorption and penetration, that some individual compounds may be more adept for either transdermal or transmucosal absorption and penetration. The difference is due in part to the particular properties associated of each tissue type, as well as the particular absorption and permeability properties associated with particular compounds. It is known that transmucosal penetration carries four times the absorption rate as transdermal penetration.

The present invention incorporates compounds that preserve the efficaciousness, effectively dilute, serve as carriers, and improve the tactile properties of the aforementioned compounds mentioned above. In one embodiment of the present invention, the topical formulation utilizes at least one compound from a group containing buffered water up to pH 9.0 (about 7.4 to 9.0), such as a biocompatible phosphate buffer that contains Na2HPO4 and/or K2HPO4, medium-chain triglycerides (MCT), bisabolol, and dimethyl isosorbide. The buffered and/or alkalinized water would preserve the stability of ATP, dilute active ingredients, and facilitate topical applications of the topical formulation. The medium-chain triglycerides (MCT) would be used to dilute and improve tactile properties of the topical formulation. The bisabolol has a secondary function that assists in the improvement of the tactile properties of the topical formulation. The dimethyl isosorbide additionally provides improved miscibility of the active ingredients.

The Table below shows one preferred embodiment of the present invention, which contains the essential components.

Innermost Core Composition

| Compounds | Quantity |
|---|---|
| D-ribose | 1.0 ml |
| Piperine | 10.0 mg |
| Distilled water | 1.2 ml |

In one embodiment of the present invention, the topical formulation is provided with an innermost core composition comprising a minimum collection of compounds that achieve the desired effects of the topical formulation. The innermost core composition of the topical formulation comprises the D-ribose, piperine, and distilled water. The D-ribose would enhance sensitivity and erections, while the piperine would support those factors as well as improve absorption. The distilled water would be the carrier for the formulation. The aforementioned composition can be modified and enhanced through the inclusion of some of the previously described compounds.

Core Composition

| Compounds | Quantity |
|---|---|
| D-ribose | 1.0 ml |
| Piperine | 10.0 mg |
| Adenosine Triphosphate | 15.0 mg |
| (ATP) | |
| Buffered and/or alkalilinized | 1.5 ml |
| water (up to pH 9.0) | |
| Bisabolol | 3 drops |

In one embodiment of the present invention, the innermost core composition is modified and enhanced through the inclusion of various additional compounds forming the core composition of the topical formulation. The core composition of the topical formulation comprises D-ribose, piperine, adenosine triphosphate, bisabolol, and buffered and/or alkalilinized water (up to pH 9.0).

The foregoing embodiment of the topical formulation was experimentally observed to produce the desired effects. In the current embodiment of the present invention, the D-ribose in the topical formulation is expected to immediately enhance erection and sensitivity. The D-ribose is abetted by the piperine, which enhances absorption and sensitivity through its thermogenic properties. The inclusion of ATP prolongs the desired effects of the topical formulation. The effects of ATP were experimentally observed to prolong the effects of the topical formulation for about an 18-hour period following administration. The buffered and/or alkalinized water is provided in the topical formulation as a means of preserving ATP from degradation.

Core Composition and Variations

| | Formulations | | | | |
|---|---|---|---|---|---|
| Compounds | Core | 1 | 2 | 3 | 4 |
| D-ribose | 1.0 ml | 1.0 ml | 1.0 ml | 1.0 ml | 1.0 ml |
| Piperine | 10.0 mg | 10.0 mg | 10.0 mg | 10.0 mg | 10.0 mg |
| Adenosine | | | | | |
| Triphosphate | 15.0 mg | 15.0 mg | 15.0 mg | 15.0 mg | 15.0 mg |
| (ATP) | | | | | |
| Buffered and/ | 1.5 ml | 1.5 ml | 1.5 ml | 1.5 ml | 1.5 ml |
| or alkalilinized | | | | | |
| water | | | | | |
| (up to pH 9.0) | | | | | |
| Bisabolol | 2 drops | 2 drops | — | 3 drops | 3 |
| | | | | | drops |
| Medium-chain | — | 2 drops | — | 3 drops | |
| triglycerides | | | | | |
| (MCT) | | | | | |
| Nicotinamide | — | — | 30.0 mg | — | — |
| riboside | | | | | |
| Creatine | — | — | — | 100 mg | |
| Dimethyl | — | — | 2 drops | — | — |
| isosorbide | | | | | |

In one embodiment of the present invention, the core composition is modified through the inclusions various compounds. The additional compounds are provided as a means of enhancing or as an alternative means of achieving the same desired effects of the topical formulation. In one embodiment of the present invention, the bisabolol, medium-chain triglycerides, nicotinamide riboside, and creatine are included to form variations of the core composition.

Alternative Core Composition

| Compounds | Quantity |
|---|---|
| Reduced nicotinamide adenine | 5.0 mg |
| dinucleotide (NADH) | |
| Dimethyl isosorbide | 2 drops |
| Buffered and/or alkalilinized water | 1.0 ml |
| (up to pH 9.0) | |

In one embodiment of the present invention, the topical formulation is provided with an alternative core composition that primarily differs by the inclusion of the reduced form of nicotinamide adenine dinucleotide (NADH).

Secondary Core Composition

| Compounds | Quantity |
|---|---|
| Reduced nicotinamide adenine | 1.0 ml |
| dinucleotide (NADH) | |
| Nicotinamide riboside | 10.0 mg |
| Distilled water | 1.5 ml |
| Bisabolol | 2 drops |

The foregoing alternative core composition is utilized to form a secondary core composition. The secondary core composition is distinguished from the alternative core composition by the inclusion of nicotinamide riboside. The secondary core composition relies on a synergistic relationship between the nicotinamide riboside and NADH. The synergistic relationship between nicotinamide riboside and NADH enhances erections and could possibly reduce a user's refractory period. Bisabolol and dimethyl isosorbide are incorporated into the secondary composition as penetration enhancers.

Secondary Core Composition Variations

| | | Formulations | | |
|---|---|---|---|---|
| Compounds | Core | 1 | 2 | 3 |
| Reduced nicotinamide | 5.0 mg | 5.0 mg | 5.0 mg | 5.0 mg |
| adenine dinucleotide | | | | |
| (NADH) | | | | |
| Nicotinamide riboside | 30.0 mg | 30.0 mg | — | 30.0 mg |
| Bisabolol | 3 drops | 3 drops | 3 drops | — |
| Buffered and/or | 1.0 ml | 1.0 ml | 1.5 ml | 1.0 ml |
| alkalinized water (up | | | | |
| to pH 9.0) | | | | |
| Creatine | — | 150 mg | — | — |
| D-ribose | — | — | 1.0 ml | — |
| Piperine | — | — | 10.0 mg | — |
| Medium-chain | — | — | 2 drops | — |
| triglycerides | | | | |
| (MCT) | | | | |
| Dimethyl isosorbide | — | — | | 2 drops |
| Adenosine | — | — | 15.0 mg | — |
| triphosphate | | | | |
| (ATP) | | | | |

In one embodiment of the present invention, the secondary core composition is modified using creatine, D-ribose, piperine, MCT, dimethyl isosorbide, and ATP, to create variations of the secondary core composition. The variation of the secondary core composition provides additional means of achieving the same or improved function of the topical formulation.

Additional Compositions

| | Formulations | | |
|---|---|---|---|
| Compounds | 1 | 2 | 3 |
| D-ribose | 1.0 ml | 1.0 ml | 1.0 ml |
| Piperine | 10.0 mg | 15.0 mg | 10.0 mg |
| Adenosine | 15.0 mg | 15.0 mg | 15.0 mg |
| triphosphate | | | |
| (ATP) | | | |
| Buffered and/or | | | |
| alkalilinized water (up | 1.5 ml | 1.5 ml | 1.7 ml |
| to pH 9.0) | | | |
| Bisabolol | 3 drops | 3 drops | 3 drops |
| Medium-chain | | | |
| triglycerides | — | — | 2 drops |
| (MCT) | | | |
| Reduced nicotinamide | — | — | 5.0 mg |
| adenine dinucleotide | | | |
| (NADH) | | | |
| Nicotinamide riboside | — | — | 30.0 mg |
| Creatine | — | — | 150 mg |
| Dimethyl isosorbide | — | — | 2 drops |

The present invention may provide additional formulations that include compounds that are known to enhance sexual function and performance. In an additional embodiment of the invention, the topical formulation may include the following compounds individually or in combination selected from the group containing vitamin C (as ascorbic acid, ascorbyl palmitate, or magnesium ascorbyl phosphate), N-acetyl L-cysteine, and glutathione.

Vitamin C (ascorbic acid) is known to support vascular tone and protect nerves. Vitamin C as ascorbyl palmitate may support endothelial function and/or absorption of the composition. Magnesium ascorbyl phosphate, vitamin C may support vasodilation through its magnesium component and its potential contribution to cellular phosphate reservoirs. N-acetyl L-cysteine is an amino acid that is a precursor to hydrogen sulfide, a gas involved endothelially in vasodilation. As well, cysteine is a precursor to glutathione (primarily produced in the liver, but capable of being produced by all cells in the body).

In addition, the present invention may also contains a few other components that would be helpful to enhance the efficacy of the present invention.

The various forms of coQ10, such as ubiquinone and ubiquinol also exhibit a functionality to support mitochondrial function, yet seems not functional in the present invention. However, one form of coQ10, which is bound to a lipophilic cation to facilitate absorption into the negatively-charged mitochondrial matrix, does seem to add benefits. It has been used as about 0.8 mg per dose.

Glycerin, in a small amount, such as one drop per dose, is able to add more substance to the otherwise watery serum, and it potentially has a biological role as an energy store.

The water base of the present invention may be an humic and fulvic colloidal mineral water solution with a pH around 9.

Another additional component is glutathione, a key antioxidant (primarily produced in the liver, but capable of being produced by all cells in the body) which has been shown to contribute to nerve function where nerves are stressed by high levels of reactive oxygen species (ROS), with ROS burden increased by up-regulated metabolic activity, as one would expect during prolonged stimulation. Glutathione is used in the present invention at 20 mg per dose.

The present invention may contain a liquid fraction (in a vial) and a powder fraction (in a small plastic bag). The two are mixed right before use (mainly to assure efficacy of unstable-in-solution ATP). Ingredients of the liquid fraction are: water, bisabolol, glycerin, fulvic and humic colloidal minerals. Ingredients of the powder fraction are: D-ribose, ATP, nicotinamide riboside, mitoquinone mesylate, and piperine.

Although the invention has been described in relation to its specific embodiments, it is to be understood that many other possible modifications and variations can be made without departing from the spirit and scope of the invention as herein described.

What is claimed is:

1. A topical formulation for enhancing sensation and function, comprising
a component or component combination for providing energy and supporting energy production in nerve and muscle cells;
a component or component combination for stimulating nerves;
a component or component combination for enhancing blood flow and vasodilating;
a component or component combination for assisting dermal and mucosal absorption;
one or a combination of additives; and
a carrier.

2. The topical formulation for enhancing sensation and function according to claim 1, comprising:
the component or component combination for providing energy and supporting energy production being selected from the group consisting of D-ribose, adenosine triphosphate (ATP), a reduced from of nicotinamide adenine dinucleotide (NADH), nicotinamide riboside, and a combination thereof.

3. The topical formulation for enhancing sensation and function according to claim 1, comprising:
the component or component combination for stimulating nerve is piperine.

4. The topical formulation for enhancing sensation and function according to claim 1, comprising:
the component or component combination for enhancing blood flow and vasodilating being selected from the group consisting of ATP, NADH, dimethyl isosorbide, a black pepper extract, and a combination thereof.

5. The topical formulation for enhancing sensation and function according to claim 1, comprising:
the component or component combination for assisting dermal and mucosal absorption being selected from the group consisting of piperine, bisabolol, dimethyl isosorbide, a medium-chain triglyceride, and a combination thereof.

6. The topical formulation for enhancing sensation and function according to claim 1, comprising:
the carrier being selected from the group consisting of a buffered water with a pH value within a range from 7.4 to 9.0, a medium-chain triglyceride, bisabolol, and dimethyl isosorbide.

7. The topical formulation for enhancing sensation and function according to claim 6, comprising:
the buffered water is a biocompatible aqueous phosphate buffer.

8. The topical formulation for enhancing sensation and function according to claim 1, comprising:
D-ribose;
piperine;
ATP; and
a buffered water having a pH value within a range from 7.4 to 9.0.

9. The topical formulation for enhancing sensation and function according to claim 8, wherein the formulation comprises 1.0 ml of D-ribose, 10.0 mg of piperine and 1.2 ml of distilled water.

10. The topical formulation for enhancing sensation and function according to claim 1, comprising:
D-ribose;
piperine;
ATP;
bisabolol; and
a buffered water having a pH value within a range from 7.4 to 9.0.

11. The topical formulation for enhancing sensation and function according to claim 10, wherein the formulation comprises 1.0 ml of D-ribose, 10.0 mg of piperine, 15.0 mg of ATP, 3 drops of bisabolol and 1.5 ml of a buffered water having a pH value within a range from 7.4 to 9.0.

12. The topical formulation for enhancing sensation and function according to claim 10, wherein the formulation further comprises a medium-chain triglyceride.

13. The topical formulation for enhancing sensation and function according to claim 10, wherein the formulation further comprises nicotinamide riboside.

14. The topical formulation for enhancing sensation and function according to claim 10, wherein the formulation further comprises a medium-chain triglyceride and nicotinamide riboside.

15. The topical formulation for enhancing sensation and function according to claim 1, comprising:
NADH;
dimethyl isosorbide; and
a distilled water.

16. The topical formulation for enhancing sensation and function according to claim 15, wherein the formulation further comprises bisabolol.

17. The topical formulation for enhancing sensation and function according to claim 16, wherein the formulation further comprises D-ribose, piperine, and a medium-chain triglyceride.

18. The topical formulation for enhancing sensation and function according to claim 15, wherein the formulation further comprises dimethyl isosorbide.

19. The topical formulation for enhancing sensation and function according to claim 1, wherein the formulation further comprises vitamin C and N-acetyl L-cysteine.

20. The topical formulation for enhancing sensation and function according to claim 1, wherein the formulation further comprises humic and fulvic colloidal minerals.
